(19) United States
(12) Patent Application Publication
(10) Pub. No.: US 2019/0008675 A1
(43) Pub. Date: Jan. 10, 2019

(54) BIOMETRICALLY RESPONSIVE ACTIVATION SYSTEM

(71) Applicant: JAMES KELLEY, HOUSTON, TX (US)

(72) Inventor: JAMES KELLEY, HOUSTON, TX (US)

(21) Appl. No.: 15/642,087

(22) Filed: Jul. 5, 2017

(51) Int. Cl. A61F 7/00

(52) U.S. Cl. CPC A61F 7/007; A61F 2007/0052; A61B 5/053; A61F 2007/0093; A61F 2007/0077

(57) ABSTRACT

A system and method for detecting the onset and conclusion of a hot flash is described. The system is configured to automatically operate a cooling device to minimize the discomfort and effects of the hot flash on a user. The system includes a wearable device and a power control device. The wearable device measures conductance across the skin to detect hot flashes. When detected, the wearable device communicates with the power control device to activate a cooling device. The cooling device is deactivated when conductance readings detect the ending of the hot flash. The automatic operation of the cooling device allows the user to get better rest by minimizing the effects of the hot flash.

### BACKGROUND

### 1. Field of the Invention

The present application relates to an automatic cooling system, and more particularly to a system that detects elevated temperature levels of a human body and automatically initiates a device to cool the same human body.

### 2. Description of Related Art

Human bodies are exothermic in that they produce their own body heat. Body temperatures can fluctuate for any number of reasons, including exercise, resting, medical conditions, and illness to name a few. Instances may arise where an individual would desire assistance in cooling off their body. For example, someone working outside in elevated temperatures may come inside to cool off. Fans may be used to produce an airflow across the skin to assist in an evaporative cooling effect. In another example, a female body going through menopause may experience hot flashes where the temperature of the body fluctuates to elevated levels. Between these examples, it is understood that sometimes the changes in body temperature occur as a result of intentional activities, knowingly to an individual; and also may occur naturally (menopause) without knowledge to an individual, while sleeping for example.

With respect to menopause, an individual experiences fluctuations in body temperature between a typical baseline and that of an elevated temperature. These fluctuations occur naturally without initiation of the individual. These elevated temperatures cause discomfort. When awake, the individual can seek comfort by moving to a cooler area or sitting near a cooling device. However, when the individual is not conscious or is sleeping, the individual is not aware sufficiently to get relief. Often the temperature flashes result in waking the individual during the night, resulting in reduced sleep.

Currently, individuals experiencing hot flashes/night sweats try to regulate their temperature at night by adjusting covers. Fans that are used to maintain a cooling effect end up running all night. They assist during a hot flash/night sweat but then make the individual too cold when the hot flash/night sweat finishes. Changes in wardrobe, layered bedding, and regulating the air temperature all have disadvantages. For example, cooling the air increases costs. Furthermore, any route taken is typically done to accommodate a single temperature condition but is not responsive to the entire fluctuation of temperatures. Therefore an individual is either too hot or too cold, each resulting in discomfort and loss of sleep and rest.

Although strides have been made to comfort individuals during hot flashes/night sweats at night, shortcomings remain. A responsive cooling system is needed that can automatically adapt to the particular temperature levels in real time so as to selectively generate a cooling effect on the individual.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a schematic of a biometrically responsive cooling system according to an embodiment of the present application.

FIG. 2 is a schematic of an exemplary electronic device used in the biometrically responsive cooling system of FIG. 1.

FIG. 3 is a chart of the function of the biometrically responsive cooling system of FIG. 1.

FIG. 4 is an alternative embodiment of the biometrically responsive cooling system of FIG. 1.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system and method in accordance with the present application overcomes one or more of the above-discussed problems commonly associated with traditional security devices for doors. In particular, the biometrically responsive cooling system of the present application is configured to regulate the operation of a cooling device so as to selectively initiate a cooling effect upon an individual in response to a detected temperature fluctuation of the user's body. The system is configured to monitor biometric conditions of the user. Use of the system is designed to minimize discomfort during the night so as to permit the user better rest despite the occurrence of hot flashes/night sweats. These and other unique features of the device are discussed below and illustrated in the accompanying drawings.

The system and method will be understood, both as to its structure and operation, from the accompanying drawings, taken in conjunction with the accompanying description. Several embodiments of the device may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The system and method of the present application is illustrated in the associated drawings. The device includes a sensor configured to be worn by a user and measure conductance levels across the skin. As a body experiences a hot flash/night sweat, or an increase in temperature, the moisture level across the skin changes thereby affecting the conductance readings of the sensor. The system includes a processor configured to capture and analyze the conductance readings and selectively generate one or more types of command data for reception by a power control device. The power control device selectively regulates power flowing to a cooling device in response to the command data from the processor. The cooling device is turned on and off during the night as needed automatically in response to the conditions of the user. Additional features and functions of the device are illustrated and discussed below.

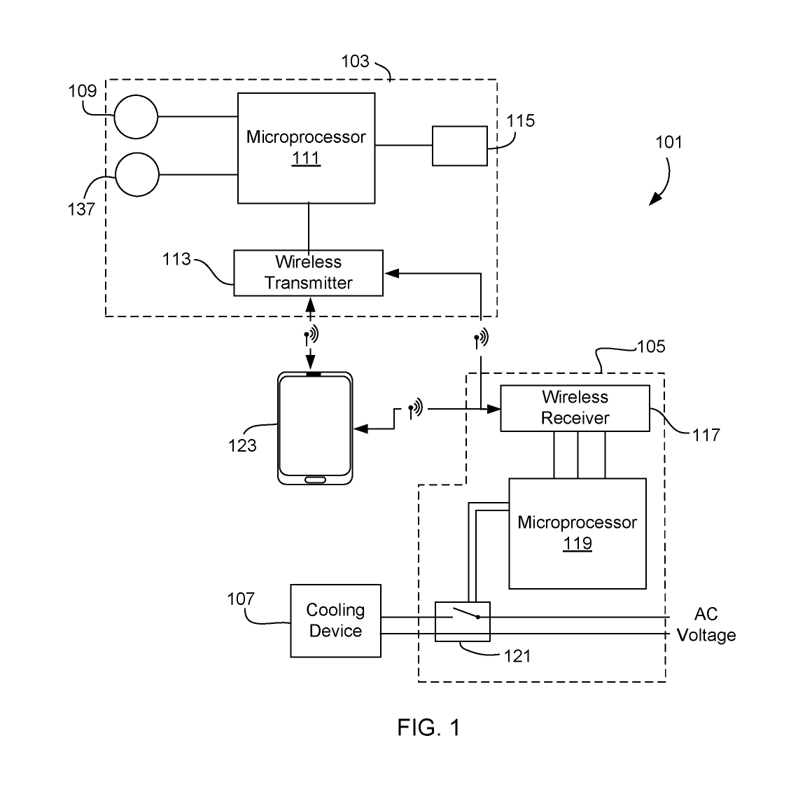

Referring now to the drawings wherein like reference characters identify corresponding or similar elements in form and function throughout the several views. FIG. 1 illustrates a schematic of a biometrically responsive cooling system 101 according to an embodiment of the present application. System 101 includes a wearable device 103 and a power control device 105. Wearable device 103 is configured to be worn by a user when resting or sleeping. Device 103 is configured to monitor biometric data of the user and selectively communicate with the power control device 105 as a spike in temperature of the user is identified. Control device 105 is configured to regulate power to a cooling device 107 (i.e. a fan) in response to communication from device 103. By regulating power to cooling device 107, a cooling effect is provided to the user automatically at the times of elevated temperatures of the body and automatically withdrawn when the elevated temperatures are finished. In this manner the two devices 103 and 105 work to provide comfort to the user without additional input from the user.

Wearable device 103 includes a sensor 109, a processor 111, a transmitter 113, and a power supply 115. Sensor 109 is configured to measure conductance across a surface of the skin of the user. Sensor 109 is placed in contact with the skin and uses an electrical current to detect variations in conductance levels on the skin. A human body naturally operates within a typical range of temperatures when at rest. Although some variations can be realized, typically the variations in the range of temperatures is fairly minimal. As the body begins to experience elevated temperatures, moisture is formed across the surface of the skin to naturally cool the body. As moisture levels increase, conductance levels also increase as moisture is a great conductor. Sensor 109 is configured to transmit a signal representing the level of conductance to processor 111.

For purposes of this application, it is understood that the term “hot flash” is an elevated temperature as a result of menopause experienced during the day. Elevated temperatures experienced at night from menopause are typically termed “night sweats”. For purposes herein, these two terms will be used interchangeably and referred to generally by “hot flash”. System 101 is ideally suited for operation during the night when the user is sleeping.

Processor 111 is configured to receive the signal representing the level of conductance from sensor 109. Processor 111 is configured to store and analyze the data in a manner that detects hot flashes or elevated temperatures in the human body. Processor 111 accomplishes this by generating or compiling a real time reading of conductance levels over time. Processor 111 also generates or compiles a delayed reading of conductance levels over time. The delayed reading of conductance levels is identical to the real time reading but merely delayed by a given time frame. Processor 111 is configured to analyze these two levels over time in an effort to detect sudden temperature increases. When an elevated temperature level is detected, processor 111 generates a first command data.

Transmitter 113 is in electrical communication with processor 111 and is configured to transmit the first command data. Transmissions of command data are done wirelessly but is understood that wire communications are possible and permitted to work herein. Power supply 115 is in communication with the various sensors and elements in wearable device 103 and is configured to provide power for their operation. Power supply 115 is ideally rechargeable to permit continuous and repeated uses.

Power control device 105 includes a receiver 117, a processor 119, and a relay 121. Device 105 is located in a remote location from that of device 103. For example, device 105 may be configured to plug into an electrical outlet, having a male set of connections and also a female plug outlet for the attachment of cooling device 107. In this manner, device 105 is configured to act between cooling device 107 and A/C voltage provided from the outlet. Device 105 may be a compact plug-in device or may be incorporated into a bed set for example wherein a cord and plug are used to plug into the wall outlet and cooling device 107 plugs into a separate location on device 105, distal from the wall outlet. It is understood that as device 105 is plugged into a wall outlet, or in communication so as to receive power from an A/C voltage source, that a D/C power supply is not necessary. It is conceived that device 105 may operate with either an A/C power source or via a D/C power supply in other embodiments. For example, the D/C power supply could act as a power backup in case of A/C power loss.

Receiver 117 is configured to receive first command data from transmitter 113 and pass that data on to processor 119. Processor 119 is configured to automatically regulate the flow of current through relay 121 in response to the first command data. Relay 121 operates between an open and closed position. When open, current is not permitted to pass to cooling device 107. When closed, relay 121 permits current to pass to cooling device 107. Cooling device 107 may therefore be in a continuously on configuration and let the device 105 regulate when power is received. When power is received, device 107 is on. When there is no power, device 107 stops working.

Cooling device 107 is designed to provide a cooling effect to the user. A simple example for this is where device 107 is a fan. Other types of devices are possible, such as cooling blankets for example. Device 107 is not limited herein to a fan.

System 101 may also include a personal electronic device 123. Device 123 is configured to receive and relay command data from device 103 to device 105. In selected embodiments, device 123 may replace processors 111 and/or 119. An additional benefit of using device 123 is that it may include application software for the storage, viewing, analyzing, and manipulation of data. For example, data related to conductivity readings, time stamps, and system performance can be retained and accessed by a user. Additionally, device 123 is able to communicate with remote internet based storage facilities via the cloud.

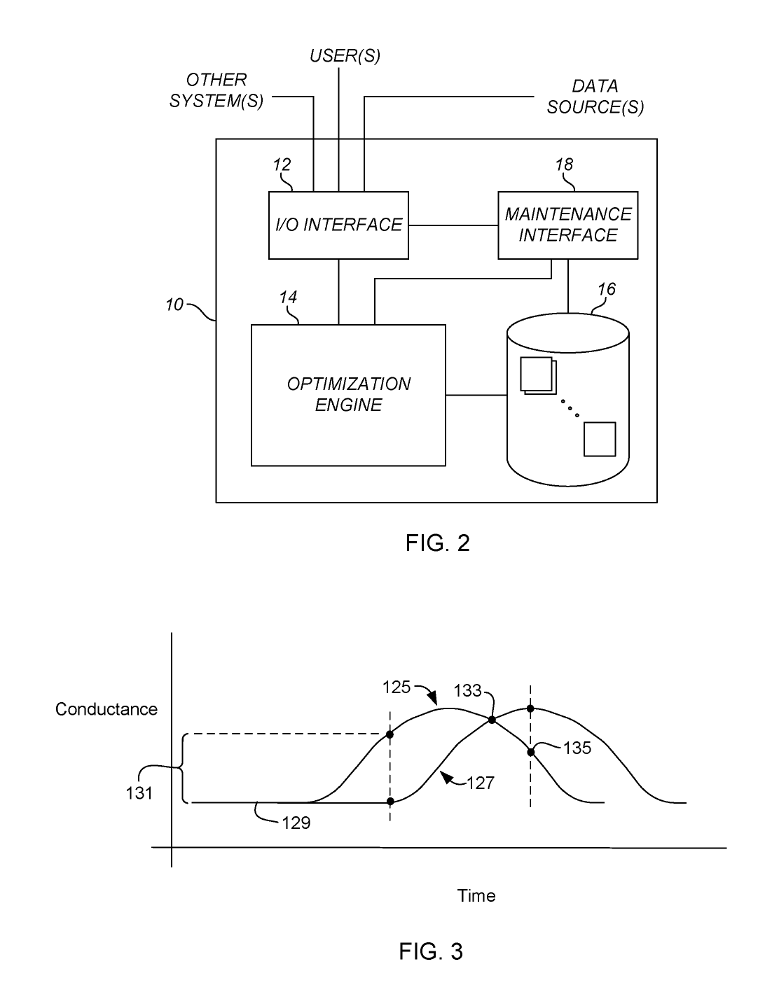

Referring now also to FIG. 2 in the drawings, a schematic of an exemplary electronic device 10 used in the biometrically responsive cooling system of FIG. 1 is illustrated. FIG. 2 illustrates an exemplary electronic device 10 that may be incorporated within or represent devices 103, 105, and 123.

The device 10 includes an input/output (I/O) interface 12, a processor 14, a database 16, and a maintenance interface 18. Alternative embodiments can combine or distribute the input/output (I/O) interface 12, processor 14, database 16, and maintenance interface 18 as desired. Embodiments of the device 10 can include one or more computers that include one or more processors and memories configured for performing tasks described herein below. This can include, for example, a computer having a central processing unit (CPU) and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while the exemplary embodiment is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example computer workstations, that are suitable for also performing other tasks. Furthermore the computers may use transitory and non-transitory forms of computer-readable media. Non-transitory computer-readable media is to be interpreted to comprise all computer-readable media, with the sole exception of being a transitory, propagating signal.

The I/O interface 12 provides a communication link between external users, systems, and data sources and components of the device 10. The I/O interface 12 can be configured for allowing one or more users to input information to the device 10 via any known input device. Examples can include a keyboard, mouse, touch screen, microphone, and/or any other desired input device. The I/O interface 12 can be configured for allowing one or more users to receive information output from the device 10 via any known output device. Examples can include a display monitor, a printer, a speaker, and/or any other desired output device. The I/O interface 12 can be configured for allowing other systems to communicate with the device 10. For example, the I/O interface 12 can allow one or more remote computer(s) to access information, input information, and/or remotely instruct the device 10 to perform one or more of the tasks described herein. The I/O interface 12 can be configured for allowing communication with one or more remote data sources. For example, the I/O interface 12 can allow one or more remote data source(s) to access information, input information, and/or remotely instruct the device 10 to perform one or more of the tasks described herein.

The database 16 provides persistent data storage for device 10. While the term “database” is primarily used, a memory or other suitable data storage arrangement may provide the functionality of the database 16. In alternative embodiments, the database 16 can be integral to or separate from the device 10 and can operate on one or more computers. The database 16 preferably provides non-volatile data storage for any information suitable to support the operation of the device 10.

The maintenance interface 18 is configured to allow users to maintain desired operation of the device 10. In some embodiments, the maintenance interface 18 can be configured to allow for reviewing and/or revising the data stored in the database 16 and/or performing any suitable administrative tasks commonly associated with database management. This can include, for example, updating database management software, revising security settings, and/or performing data backup operations. In some embodiments, the maintenance interface 18 can be configured to allow for maintenance of the processor 14 and/or the I/O interface 12. This can include, for example, software updates and/or administrative tasks such as security management and/or adjustment of certain tolerance settings.

Referring now also to FIG. 3 in the drawings, a chart of the function of the biometrically responsive cooling system of FIG. 1 is illustrated. As stated previously, processor 111 is configured to process conductance levels over time and analyze them to determine the occurrence of a hot flash and its eventual conclusion so as to properly operate cooling device 107. Processor 111 is configured to generate a baseline conductance reading level. The baseline conductance reading is an average of the normal conductance level fluctuations as detected by sensor 109. The baseline is formed from normal temperature levels of the user.

Processor 111 generates or compiles a real time reading of conductance levels over time. Processor 111 also generates or compiles a delayed reading of conductance levels over time. The delayed reading of conductance levels is a copy of the real time readings, but is merely delayed by a given time interval (i.e. 30 seconds, 1 minute, or 5 minutes for example). Processor 111 is configured to analyze these two levels over time in an effort to detect sudden temperature increases. FIG. 3 illustrates a chart showing real time readings 125 and delayed readings 127.

Processor 111 stores conductance readings at a selected time interval. Device 103 is configured to compare the readings to determine the start of a hot flash. This may be done in a number of ways. For example, real time readings 125 may be compared to baseline readings 129. When levels differentiate by a prescribed amount, device 103 recognized the start of the hot flash. By monitoring the rate of change of real time readings 125 as compared to that of baseline 129, device 103 is able to determine the initiation and conclusion of a hot flash.

In another example, processor 111 compares the readings from the real time readings 125 to that of the delayed readings 127. In this situation, the differentiation between the two readings 125 and 127 trigger the recognition of the onset of a hot flash. The differentiation 131 can be selectively prescribed by a user to adjust for the user and severity of hot flashes. At this time, the first command data is transmitted to device 105 for turning on the cooling device 107.

As time progresses, node 133 is reached where the conductance readings from real time readings 125 and delayed readings 127 cross paths. At this moment, device 103 detects the beginning of the end of the elevated temperatures experienced by the user. Device 103 transmits a second command data to device 105 to begin a delayed shut down. This delayed shut down is a time interval in which the cooling device 107 will continue to operate until device 105 opens delay 121 and cuts off power.

At node 135, device 103 detects the differentiation between the real time readings 125 are lower than that of the delayed readings 127 by a predetermined amount such that device 103 may optionally send a third command data to device 105 to shut off power to cooling device 105 regardless of the time left in the delay. At node 135, the hot flash is considered ended. Alternatively, at node 135, device 105 may continue with the delay and shut power off to cooling device 107 at the conclusion of the delayed time period. As seen, by monitoring and comparing conductance readings over time, system 101 is able to automatically identify the beginning and ending of a hot flash and correspondingly activate and deactivate a cooling device. By maintaining a more comfortable environment for the user, the user is able to get more rest and minimize the heat fluctuations of the user.

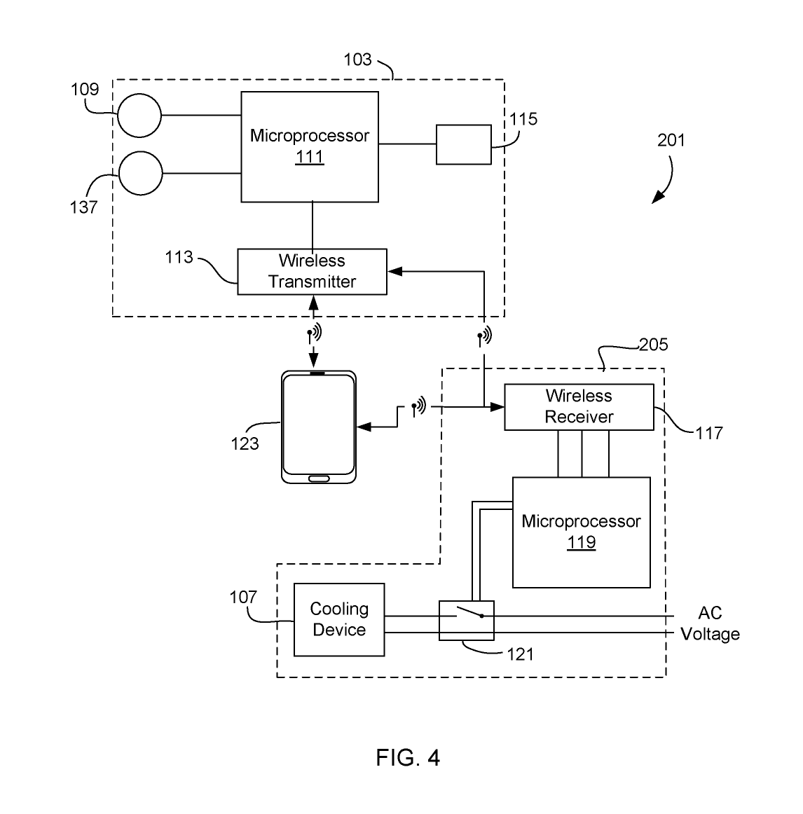

Referring now also to FIG. 4 in the drawings, an alternative embodiment of the biometrically responsive cooling system 101 is illustrated. Biometrically responsive cooling system 201 is similar in form and function to that of system 101 except as herein noted. Like references refer to corresponding and identical members between the two systems. System 201 is different in that cooling device 107 is included within power control device 105, thereby forming power control device 205. By including them together, assemblies are considered feasible where a portable fan can be advanced to include all of device 105. Additionally, device 105 can be any other type of assembly that also then incorporated a cooling device. For example, a bed can have a power cord that includes an internal cooling device to activate at selected times. This bed could be synced with device 103. Alternatively, the cooling device could merely plug into the bed and work together as well. Other possibilities exist.

It is worth noting that system 101 may further include a secondary sensor 137 in communication with device 103. Sensor 137 is a biometric sensor configured to capture biometric data of the user during the night or resting period. Such biometric data could include: hear rate, oxygen level, and so forth. Other biometric sensors may also be synced with device 103.

The current application has many advantages over the prior art including at least the following: (1) a wearable device configured to detect the onset and conclusion of hot flashes based upon conductance readings on the surface of the skin; (2) the automatic activation of a cooling device when a hot flash begins; (3) ability to turn off the cooling device automatically at the conclusion of the hot flash; and (4) ability to integrate the functions of a personal electronic device (i.e. cell phone, tablet . . . ) with a software application to personalize the parameters of the system.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A biometrically responsive cooling system, comprising:
a first sensor configured to measure conductance across a surface, the first sensor configured to transmit a signal representing the level of conductance;
a processor configured to receive the signal representing the level of conductance, the processor configured to generate a real time reading of conductance levels over time, the processor configured to generate a delayed reading of conductance levels over time, the processor configured to compare the real time reading to the delayed reading to selectively generate and send a first command data;
a power control device configured to receive the command data and selectively regulate the flow of current through a relay in response to the command data; and
a cooling device in communication with the relay, the cooling device configured to operate in response to conductance levels from the first sensor.

2. The system of claim 1, wherein the first sensor is a conductance sensor configured to measure conductance across the surface of skin.

3. The system of claim 1, wherein the level of conductance is influenced by moisture across the surface.

4. The system of claim 1, wherein the delayed reading is a copy of the real time reading only delayed by a selected time interval.

5. The system of claim 1, wherein the processor is configured to create a baseline conductance reading, the baseline conductance reading is an average of the normal conductance level fluctuations as detected by the first sensor.

6. The system of claim 1, wherein the processor is configured to generate a first command data when the real time reading conductance level exceeds the baseline reading conductance level by a prescribed value.

7. The system of claim 1, wherein the processor is configured to generate a second command data when the real time reading conductance level is the same as the delayed reading conductance level.

8. The system of claim 7, wherein the power control device receives the second command data and initiates a delayed timer to cease the passage of current to the cooling device after a prescribed time limit has passed.

9. The system of claim 1, wherein the processor is configured to generate a third command data when the real time reading conductance level is below the delayed reading conductance level by a second prescribed value.

10. The system of claim 9, wherein the power control device receives the third command data and ceases the passage of current to the cooling device after a prescribed time limit has passed.

11. The system of claim 1, wherein the cooling device is included within the power control device.

12. The system of claim 1, wherein the processor is configured to communicate the command data through a portable electronic device.

13. The system of claim 1, further comprising:
a portable electronic device including a software application configured to store and analyze historical performance readings at least one of the processor, the first sensor, and the power control device, the portable electronic device in communication with the processor and the power control device.

14. The system of claim 1, further comprising:
a second sensor configured to capture biometric data of a user, the biometric data being transmitted to the processor.

15. The system of claim 14, wherein the biometric data is communicated to the portable electronic device.

16. The system of claim 1, wherein the first sensor is worn by a user.

17. The system of claim 1, wherein the power control device is configured to cycle power on and off to a cooling device, the cooling device configured to initiate a cooling effect on a user.

18. The system of claim 17, wherein regulating the operation of the cooling device assists in minimizing heat fluctuations of the user.
